(19) United States
(12) Patent Application Publication
(10) Pub. No.: US 2012/0136384 A1
(43) Pub. Date: May 31, 2012

(54) GRADUATED ARTERIAL DILATION BALLOON CATHETER

(75) Inventor: Douglas Pierce MacMillan, Jr., Asheville, NC (US)

(73) Assignee: SIMPLE ENDOVASCULAR, LLC, Charlotte, NC (US)

(21) Appl. No.: 12/955,982

(22) Filed: Nov. 30, 2010

(51) Int. Cl. A61M 29/02

(52) U.S. Cl. 606/194

(57) ABSTRACT

A graduated arterial dilation balloon catheter, including an elongate catheter body for being introduced into an artery, and including a inflatable treatment zone section for being positioned in a section of an artery to be treated, a first, relatively small diameter balloon positioned distally in the treatment zone section, and a second, relatively large diameter inflatable balloon positioned proximally within the treatment zone section of the catheter body in longitudinal alignment with the first balloon and with the catheter body. A first dilation lumen is provided and extends in fluid communication along the catheter body between a first dilation port and the first balloon; and a second dilation lumen is provided and extends in fluid communication along the catheter body between a second dilation port and the second balloon.

### TECHNICAL FIELD AND BACKGROUND OF THE INVENTION

This invention relates to devices and procedures intended to improve blood flow in small to medium caliber arteries, particularly in the lower extremities. This condition is known as Peripheral Artery Disease (“PAD”) and is caused by narrowed arteries that are often the result of the accumulation of atherosclerotic plaques on the interior walls of the arteries. Atherosclerosis (also known as arteriosclerotic vascular disease or ASVD) is a condition in which an artery wall thickens as the result of a build-up of fatty materials such as cholesterol. It is a syndrome affecting arterial blood vessels, a chronic inflammatory response in the walls of arteries, in large part due to the accumulation of macrophage white blood cells and promoted by low-density lipoproteins (plasma proteins that carry cholesterol and triglycerides) without adequate removal of fats and cholesterol from the macrophages by functional high density lipoproteins (HDL). It is commonly referred to as a “hardening” or “furring” of the arteries. The atheromatous plaque is divided into three distinct components, the atheroma, a nodular accumulation of a soft, flaky, yellowish material at the center of large plaques, composed of macrophages nearest the lumen of the artery, underlying areas of cholesterol crystals, and calcification at the outer base of older/more advanced lesions.

The uneven plaque accumulation and relatively small size of the arteries in the lower extremities present difficulties in efficiently and effectively ameliorating this condition.

The result of the blocked or narrowed artery is reduced blood flow to the lower legs and feet that can cause gangrene, chronic ulceration, ischemic rest pain and claudication. Presently, this condition can be treated surgically by bypassing the blockage with a natural or synthetic alternative pathway, or by a number of non-surgical minimally-invasive options, including angioplasty, stenting and atherectomy. All of these procedures have drawbacks when applied to small and medium arteries such as found in the lower legs.

For example, a number of procedures are known for enlarging arterial segments narrowed by atherosclerotic plaques.

Balloon angioplasty can be used to expand the lumen of an artery that has been compromised. A deflated balloon is introduced through an arterial puncture and advanced over a wire into the location of the narrowed arterial segment. Expansion of the balloon then enlarges the flow lumen by compressing the plaque and stretching the compliant, outer layer of the artery (adventitial layer). However, conventional balloon catheters are uniform in diameter. When a conventional balloon is inflated inside an artery, the diameter of the artery in the treated segment is stretched by balloon expansion while the arterial segments immediately adjacent are unaffected. This results in an abrupt change in transmitted pressure and diameter of the artery at opposite ends of the expanded balloon. Consequences can include flow limiting arterial dissections, and flow turbulence at these locations. Immediate or interval arterial occlusion can follow such events and diminish clinical benefit for the patient.

Stenting, which can be quite effective when used in medium to large diameter arteries such as those found in the neck, pelvis, and thighs, is problematic in smaller arteries, because it is difficult to deliver a stent that, in a collapsed state, is small enough to introduce into the artery and expand, while leaving enough open area in the expanded stent to allow proper blood flow. This is especially problematic in small caliber arteries with plaques affecting long segments.

In the case of atherectomy, longitudinal sections of plaque are cut away with a dynamic tool that is pushed through the artery. These techniques rely on relatively expensive one-time-use devices. Complications may occur when the arterial wall is injured by the cutting mechanism or loose atherosclerotic material embolizes distally causing further blockage of the artery. Furthermore, long term patency of the treated segments has been problematic in small caliber vessels.

### SUMMARY OF THE INVENTION

Therefore, it is an object of the invention to provide a wire-guided, catheter based device and procedure that is used to treat Peripheral Artery Disease at reduced cost and risk with a greater degree of patient benefit.

These and other objects and advantages of the present invention are achieved by providing an arterial dilation balloon catheter, comprising an elongate catheter body for being introduced into an artery, including a inflatable treatment zone section for being positioned in a section of an artery to be treated. A first, relatively small diameter balloon is positioned distally in the treatment zone section, and a second, relatively large diameter inflatable balloon is positioned proximally within the treatment zone section of the catheter body in longitudinal alignment with the first balloon and with the catheter body. A first dilation lumen extends in fluid communication along the catheter body between a first dilation port and the first balloon, and a second dilation lumen extends in fluid communication along the catheter body between a second dilation port and the second balloon.

According to one preferred embodiment of the invention, the catheter body includes an insertion opening for receiving a monorail guide wire.

According to another preferred embodiment of the invention, a third relatively small diameter balloon is provided, and positioned most proximally in the treatment zone section in longitudinal alignment with the first balloon, the second balloon and the catheter body. The treatment zone section of the catheter body defines a fore and aft graduated-diameter outer wall for providing a smooth, graduated reduction in expansion diameter and pressure that will be applied to the arterial wall above and below the intended arterial treatment zone.

According to another preferred embodiment of the invention, the catheter body includes an exit port for receiving a monorail guide wire communicating with a centrally-positioned guide wire lumen extending the along the length of the catheter body from tip of the device to the monorail exit port about 40 cm proximal to that tip on the catheter body.

According to another preferred embodiment of the invention, the first dilation lumen comprises a first arcuate void extending in fluid communication along the catheter body between a first dilation port and at least one fill port in a wall of the first dilation lumen in fluid communication with the first balloon for permitting injection and withdrawal of a dilation fluid. The second dilation lumen comprises a second arcuate void extending in fluid communication along the catheter body between a second dilation port and at least one fill port in a wall of the second dilation lumen in fluid communication with the second balloon for permitting injection and withdrawal of a dilation fluid.

According to another preferred embodiment of the invention, a third, relatively small diameter balloon is provided, and is positioned most proximally in the treatment zone section in longitudinal alignment with the first balloon, the second balloon and the catheter body. The treatment zone section of the catheter body defines a fore and aft tapered cylindrical wall for providing a smooth, graduated reduction in expansion diameter and pressure that will be applied to the arterial wall above and below the intended arterial treatment zone. The first and third balloons are expanded simultaneously with fluid transmitted through a contiguous fill channel within the catheter body.

According to another preferred embodiment of the invention, the small diameter balloon is adapted to be inflated with a fluid at a predetermined relatively high pressure, and the large diameter balloon is adapted to be inflated with a fluid at a predetermined relatively low pressure.

According to another preferred embodiment of the invention, the first dilation lumen comprises a first arcuate void extending in fluid communication along the catheter body between a first dilation port and a plurality of longitudinally spaced-apart fill ports in fluid communication with the first balloon, and the second dilation lumen comprises a second arcuate void extending in fluid communication along the catheter body between a second dilation port and a plurality of longitudinally spaced-apart fill ports in fluid communication with the second balloon.

According to another preferred embodiment of the invention, an arterial dilation balloon catheter is provided, and comprises an elongate catheter body for being introduced into an artery, and including a inflatable treatment zone section for being positioned in a section of an artery to be treated, and a first, relatively small diameter balloon positioned distally in the treatment zone section. A second, relatively large diameter inflatable balloon is positioned proximally in the treatment zone section of the catheter body in longitudinal alignment with the first balloon and with the catheter body, and a third, relatively small diameter balloon is positioned most proximally in the treatment zone section in longitudinal alignment with the first balloon, the second balloon and the catheter body. The treatment zone section of the catheter body defines a tapered outer wall for providing a smooth, graduated reduction in expansion diameter along the length of the treatment zone section of the catheter body. A first dilation lumen extends in fluid communication along the catheter body between a first dilation port and the first balloon and the third balloon, and includes a first arcuate void extending in fluid communication along the catheter body between a first dilation port a plurality of longitudinally spaced-apart fill ports in fluid communication with the first balloon and a plurality of longitudinally spaced-apart fill ports in fluid communication with the third balloon. A second dilation lumen extends in fluid communication along the catheter body between a second dilation port and a plurality of longitudinally spaced-apart fill ports in fluid communication with the second balloon.

According to another preferred embodiment of the invention, a method of dilating a narrowed artery is provided, comprising the steps of providing an elongate catheter body for being introduced into an artery, including a inflatable treatment zone section for being positioned in a section of an artery to be treated, a first, relatively small diameter balloon positioned distally in the treatment zone section, a second, relatively large diameter inflatable balloon positioned proximally within the treatment zone section of the catheter body in longitudinal alignment with the first balloon and with the catheter body, a first dilation lumen extending in fluid communication along the catheter body between a first dilation port and the first balloon, and a second dilation lumen extending in fluid communication along the catheter body between a second dilation port and the second balloon. The method further includes the steps of introducing the treatment zone section of the elongate catheter body into a section of an artery to be treated, inflating the first balloon with a fluid to an extent sufficient to cause the first balloon to dilate the diseased segment of artery itself so as to enlarge the effective blood flow lumen in a manner defining a smooth, graduated transition between plaque on artery walls in the section of the artery being treated and plaque on an adjacent artery wall not being treated, and inflating the second balloon with a fluid to an extent sufficient to cause the second balloon to dilate the artery in a manner defining a smooth, graduated transition between plaque on artery walls in the section of the artery being treated by the second balloon and plaque on artery walls in the section of the artery being treated by the first balloon. The method further includes the steps of deflating the first and second balloons, and withdrawing the catheter from the artery.

According to another preferred embodiment of the invention, the method includes the steps of providing a third, relatively small diameter balloon positioned most proximally in the treatment zone section in longitudinal alignment with the first balloon, the second balloon and the catheter body, wherein the treatment zone section of the catheter body defining a fore and aft-tapered wall. The third balloon is inflated with a fluid to an extent sufficient to cause the third balloon to compress and flatten arterial plaque against interior walls of the artery in a manner defining a smooth, graduated transition between plaque on artery walls in the section of the artery being treated and plaque on an adjacent artery wall not being treated and then deflating the third balloon.

According to another preferred embodiment of the invention, the method includes the step of guiding the catheter in the artery on a monorail guide wire.

According to another preferred embodiment of the invention, the method includes the steps of inflating the small diameter balloon with a fluid at a predetermined relatively high pressure, and inflating the large diameter balloon with a fluid at a predetermined relatively low pressure.

According to another preferred embodiment of the invention, the method includes the steps of inflating the first and third balloons with a liquid at a predetermined relatively high pressure, and inflating the second, large diameter balloon with a liquid at a predetermined relatively low pressure.

### BRIEF DESCRIPTION OF THE DRAWINGS

Some of the objects of the invention have been set forth above. Other objects and advantages of the invention will appear as the description of the invention proceeds when taken in conjunction with the following drawings, in which:

FIG. 1 is a overall view of a balloon catheter according to one embodiment of the invention;

FIG. 2 is an enlarged view of the distal end of the balloon catheter;

FIG. 3 is a fragmentary enlarged view of the treatment zone section of the balloon catheter;

FIG. 4 is an enlarged view of the treatment zone section of the balloon catheter;

FIG. 5 is a cross section taken along line 5-5 of FIG. 3;

FIG. 6 is a cross-section taken along lines 6-6 of FIG. 3;

FIG. 7 is a cross-section of an artery showing the treatment zone section of the balloon catheter positioned in the artery in an uninflated configuration;

FIG. 8 is a cross-section of an artery showing the treatment zone section of the balloon catheter positioned in the artery in an inflated configuration; and

FIG. 9 is a cross-section of an artery showing in a simplified, schematic form the result of the balloon angioplasty procedure after removal of the balloon catheter.

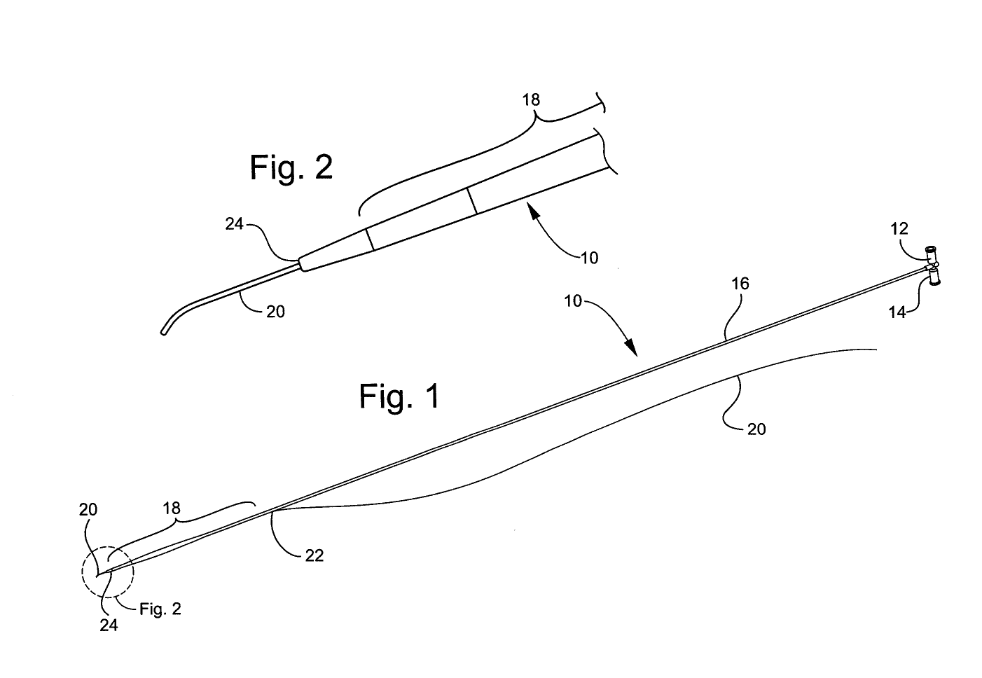

### DESCRIPTION OF THE PREFERRED EMBODIMENTS AND BEST MODE

Referring now specifically to the drawings, a overall view of a balloon catheter according one preferred embodiment of the present invention is shown generally in FIG. 1 at reference numeral 10. The balloon catheter 10 includes two entry ports 12, 14 for introducing fluids at two different pressures, as described in further detail below, and an elongate catheter body 16. A treatment zone section 18 is positioned near the distal end of the catheter body 16. A monorail guide wire 20 is introduced into the interior of the catheter body 16 through an insertion opening 22 and exits the tip of the catheter body 16 through an exit opening 24 in the distal end.

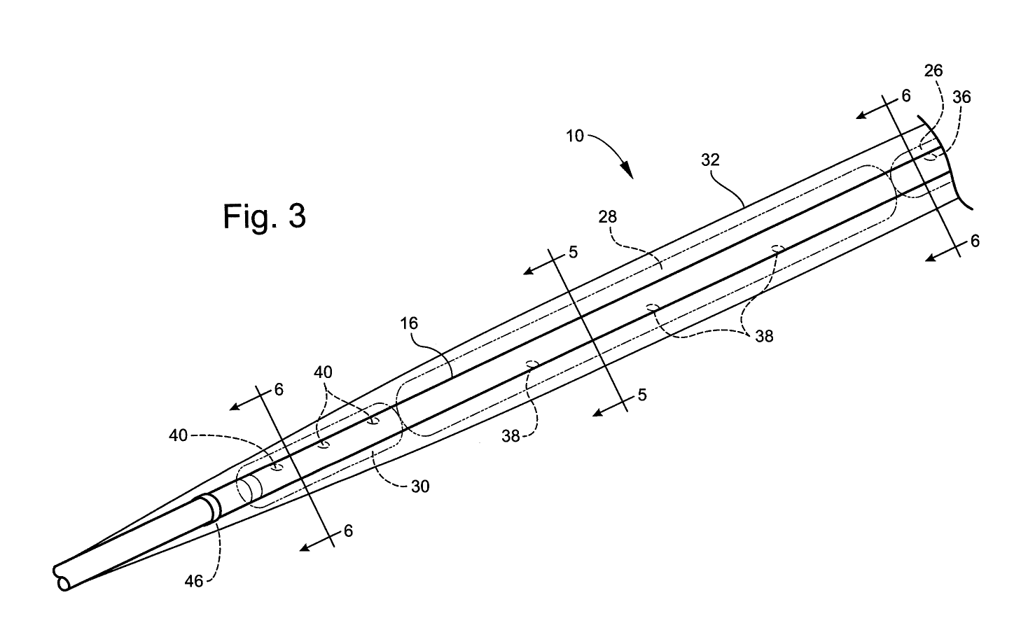

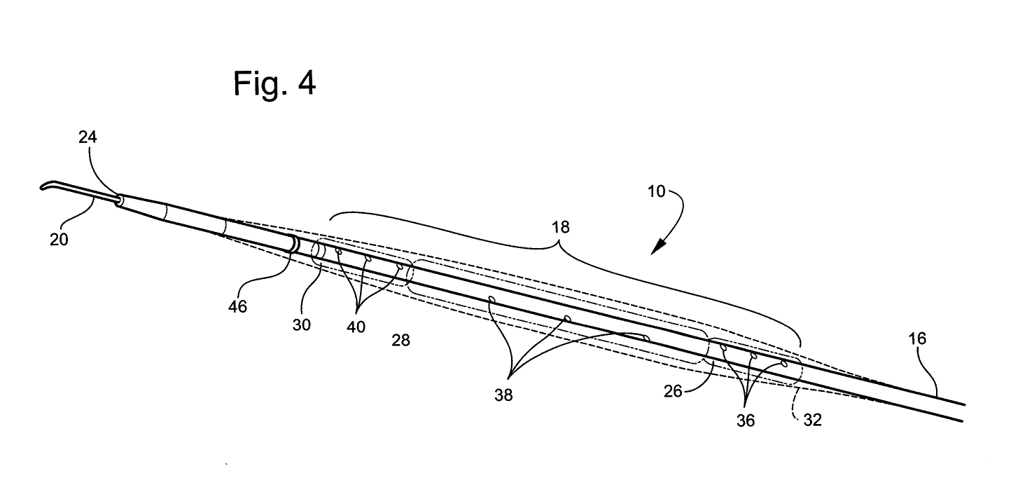

As is shown in FIGS. 3 and 4, the treatment zone section 18 includes, in this preferred embodiment, three balloons 26, 28, 30. The balloons 26, 28, 30 are formed on the outer wall of the catheter body 16, and are covered by an expansible membrane 32 that extends along the treatment zone section 18 of the catheter body 16. The expansible membrane 32 is formed of a moderately rigid polymer approved for use in the human body and is provided, as is the remainder of the exposed portions of the catheter body 16, with a hydrophilic surface with a very low coefficient of friction that permits a smooth, gradual insertion and removal of the catheter body 16.

The balloons 26 and 30 are relatively smaller than the balloon 28, and thus collectively form an overall shape within the expansible membrane 32 that has the greatest nominal diameter approximately at the middle of the length of the balloon 28, tapering in both the proximal and distal directions along the balloons 26 and 30 to a diameter approximately the same as the outer diameter of the catheter body 16. The expansible membrane 32 has sufficient elasticity to inflate and deflate as the balloons 26, 28, and 30 are inflated and deflated.

As is shown in FIGS. 3 and 4, fill ports 36 communicate for fluid flow with balloon 26, fill ports 38 communicate with balloon 28, and fill ports 40 communicate with balloon 30.

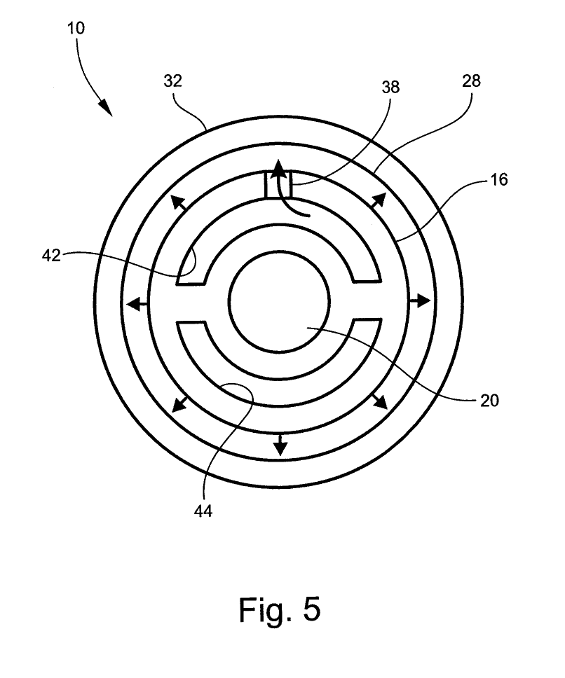

Referring now to FIGS. 5 and 6, fluid, for example, saline solution, is introduced into a pair of lumens 42 and 44 that extend down the length of the catheter body 16 between the entry ports 12, 14 and the fill ports 36, 38 and 40.

As shown in FIG. 5, lumen 42 communicates with the center, larger balloon 28 through the fill ports 38 (one shown). As saline is pumped into the balloon 28 through the fill ports 38, the expansible membrane 32 inflates. As saline is pumped out of the balloon 28, the expansible membrane 32 deflates back to a normal diameter.

Referring to FIG. 6, lumen 44 communicates with the two distal and proximal, smaller, balloons 26 and 30. Balloon 26 is shown for illustrative purposes. In a preferred embodiment, the same lumen 44 communicates with balloon 30 in the same manner. Balloon 26 (and also balloon 40) is filled through fill ports 36 and 40, respectively. As a solution of saline and iodinated contrast is pumped into the balloons 26, 30 through the fill ports 36 and 40, the expansible membrane 32 inflates. As the solution is pumped out of the balloons 26 and 30, the expansible membrane 32 deflates back to a normal diameter. Because of the differing diameters of the larger balloon 28 and the smaller balloons 26 and 30, the expansive membrane 32 expands in a long, gradual taper from the approximate center of the balloon 28 towards the distal and proximal ends of the catheter body 16.

The balloon catheter 10 includes an x-ray visible marker 46 to assist in the physician in properly locating the treatment zone 18 of the catheter body 16 at the correct location within the artery.

A preferred embodiment of the balloon catheter 10 is further exemplified according to the specifications set out below:
Diameter of catheter body 16 (1.5 mm)
Diameter of balloon 28 (uninflated) (2 mm)
Diameter of balloon 28 (inflated) (2.5-4.5 mm)
Length of balloon 28 (20-60 mm)
Diameter of balloons 26, 30 (uninflated) (2.0 mm)
Diameter of balloons 26, 30 (inflated) (2.0-3.5 mm)
Length of balloons 26, 30 (10-20 mm)
Diameter of guide wire (0.014 in)
Length of treatment zone 18 (4-10 cm)
Fluid pressure in balloon 18 at max. inflation (12 Atm)
Range of fluid pressure along length of balloon 18 at max. inflation (8-12 Atm)
Fluid pressure in balloons 26, 30 at max. inflation (16 Atm)
Range of fluid pressure along length of balloons 26, 30 at max. inflation (10-16 Atm)
Outer membrane 32
   Length, approximately 4.5-10.5 cm
   Shore A Hardness 85.

These values are merely illustrative. While balloons 26, 30 are illustrated and described as being the same length and diameter, this is not required, and either balloon may be vary in length or diameter as needed to meet particular medical requirements, for example, the diameter of the artery or the length of the blocked portion of the artery.

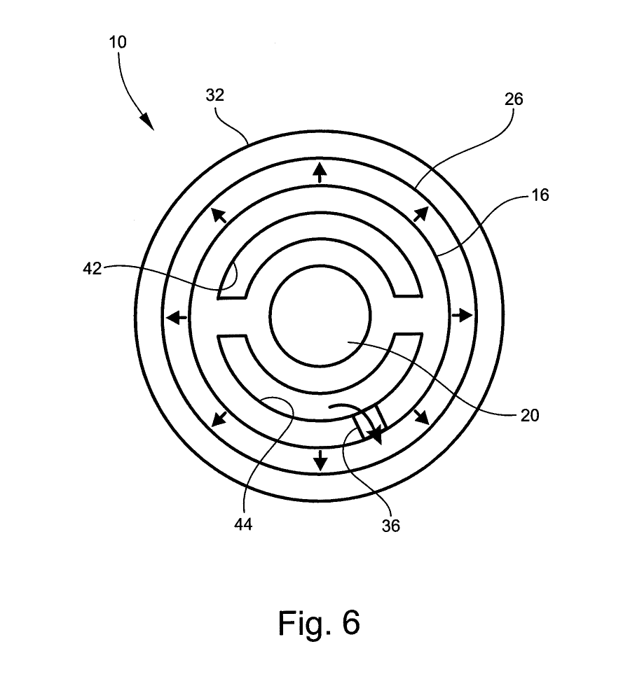

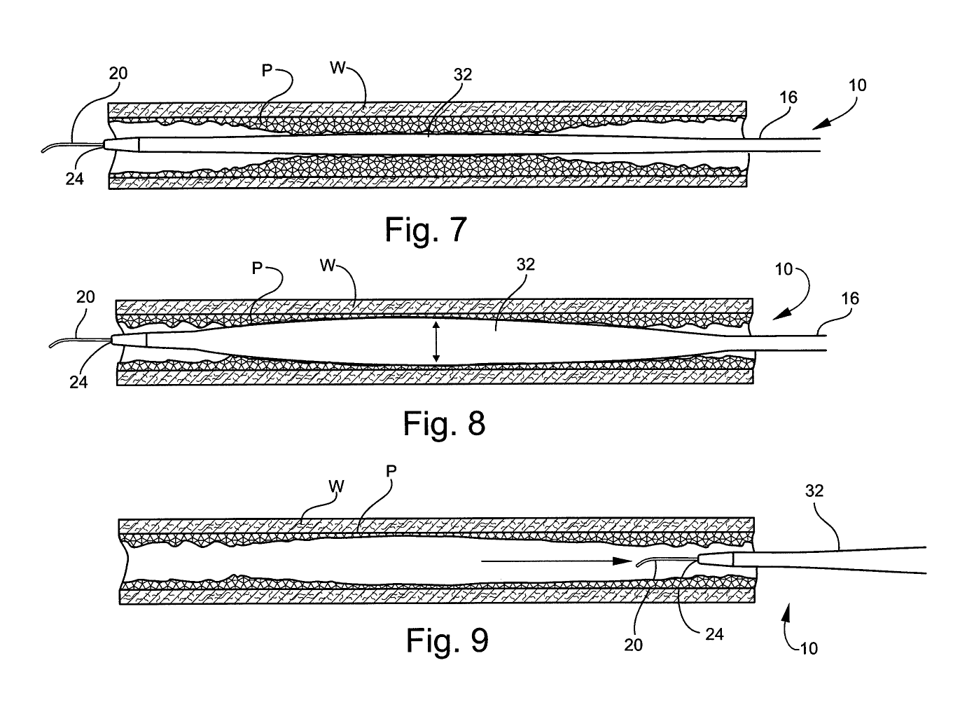

Referring now to FIG. 7, the treatment zone 18 of the balloon catheter 10 is shown, uninflated, positioned in a schematically illustrated artery, which includes an artery wall “W” and a irregular layer of plaque “P” deposited and adhered to the inner surface of the artery wall “W.” As noted above, conventional treatment with a conventional balloon catheter would result in a compaction of the plaque along the length of the balloon, but leaving a distinct and substantial radially-extending shoulder at the opposite termini of the balloon.

Referring to FIG. 8, the balloon catheter 10 is shown in its inflated configuration. Note that as with conventional balloon catheters, use of the balloon catheter 10 results in an increase in the diameter of the flow path of the artery, but this is accomplished along the length of the treatment zone with a gradual taper being applied to the plaque “P” by the tapered treatment zone 18 so that it transitions into the thickness of the plaque “P” adjacent to the treatment zone 18. The tapered shape of the treatment zone 18 also permits “flossing” of the artery wall, i.e., gently reciprocating the balloon catheter 10 within the artery during treatment to further smooth the plaque layer “P”.

An improved arterial dilation balloon catheter is described above. Various details of the invention may be changed without departing from the scope of the invention. Furthermore, the foregoing description of the preferred embodiment of the invention and best mode for practicing the invention are provided for the purpose of illustration only and not for the purpose of limitation, the invention being defined by the claims.

I claim:

1. A graduated arterial dilation balloon catheter, comprising:
(a) an elongate catheter body for being introduced into an artery, and including a inflatable treatment zone section for being positioned in a section of an artery to be treated;
(b) a first, relatively small diameter balloon positioned distally in the treatment zone section;
(c) a second, relatively large diameter inflatable balloon positioned proximally within the treatment zone section of the catheter body in longitudinal alignment with the first balloon and with the catheter body;
(d) a first dilation lumen extending in fluid communication along the catheter body between a first dilation port and the first balloon; and
(e) a second dilation lumen extending in fluid communication along the catheter body between a second dilation port and the second balloon.

2. An arterial dilation balloon catheter according to claim 1, wherein the catheter body includes an insertion opening for receiving a monorail guide wire.

3. An arterial dilation balloon catheter according to claim 1, and including a third relatively small diameter balloon positioned most proximally in the treatment zone section in longitudinal alignment with the first balloon, the second balloon and the catheter body, the treatment zone section of the catheter body defining a fore and aftgraduated-diameter outer wall for providing a smooth, graduated reduction in balloon expansion diameter along the length of the treatment zone section of the catheter body.

4. An arterial dilation balloon catheter according to claim 2, wherein the catheter body includes an insertion opening for receiving a monorail guide wire communicating with a centrally-positioned guide wire passage extending the along the length of the catheter body from the insertion opening to a guide wire exit opening at a distal tip of the catheter body.

5. An arterial dilation balloon catheter according to claim 1, wherein:
(a) the first dilation lumen comprises a first arcuate void extending in fluid communication along the catheter body between a first dilation port and at least one fill port in a wall of the first dilation lumen in fluid communication with the first balloon for permitting injection and withdrawal of a dilation fluid; and
(b) the second dilation lumen comprises a second arcuate void extending in fluid communication along the catheter body between a second dilation port and at least one fill port in a wall of the second dilation lumen in fluid communication with the second balloon for permitting injection and withdrawal of a dilation fluid.

6. An arterial dilation balloon catheter according to claim 5, and including a third, relatively small diameter balloon positioned most proximally in the treatment zone section in longitudinal alignment with the first balloon, the second balloon and the catheter body, the treatment zone section of the catheter body defining a fore and afttapered cylindrical wall for providing a smooth, graduated reduction in balloon expansion diameter along the length of the treatment zone section of the catheter body, wherein the first dilation lumen communicates for fluid flow with the third balloon through a fill port in fluid communication with the third balloon.

7. An arterial dilation balloon catheter according to claim 1, wherein the small diameter balloon is adapted to be inflated with a fluid at a predetermined relatively high pressure, and the large diameter balloon is adapted to be inflated with a fluid at a predetermined relatively low pressure.

8. An arterial dilation balloon catheter according to claim 1 wherein:
(a) the first dilation lumen comprises a first arcuate void extending in fluid communication along the catheter body between a first dilation port and a plurality of longitudinally spaced-apart fill ports in fluid communication with the first balloon; and
(b) the second dilation lumen comprises a second arcuate void extending in fluid communication along the catheter body between a second dilation port and a plurality of longitudinally spaced-apart fill ports in fluid communication with the second balloon.

9. An arterial dilation balloon catheter, comprising:
(a) an elongate catheter body for being introduced into an artery, and including a inflatable treatment zone section for being positioned in a section of an artery to be treated;
(b) a first, relatively small diameter balloon positioned distally in the treatment zone section;
(c) a second, relatively large diameter inflatable balloon positioned proximally in the treatment zone section of the catheter body in longitudinal alignment with the first balloon and with the catheter body;
(d) a third relatively small diameter balloon positioned most proximally in the treatment zone section in longitudinal alignment with the first balloon, the second balloon and the catheter body, the treatment zone section of the catheter body defining a tapered outer wall for providing a smooth, graduated reduction in atherosclerotic material thickness along the length of the treatment zone section of the catheter body.
(e) a first dilation lumen extending in fluid communication along the catheter body between a first dilation port and the first balloon and the third balloon, and including a first arcuate void extending in fluid communication along the catheter body between a first dilation port and a plurality of longitudinally spaced-apart fill ports in fluid communication with the first balloon and a plurality of longitudinally spaced-apart fill ports in fluid communication with the third balloon; and
(f) a second dilation lumen extending in fluid communication along the catheter body between a second dilation port and a plurality of longitudinally spaced apart fill ports in fluid communication with the second balloon.

10. An arterial dilation balloon catheter according to claim 1, wherein the catheter body includes a moderately rigid, hydrophilic expansible membrane enclosing the first, second and third balloons proportioned to further create the desired graduated contour supported by the enclosed balloons.

11. A method of dilating a narrowed artery, comprising the steps of:
(a) providing an elongate catheter body for being introduced into an artery, and including a inflatable treatment zone section for being positioned in a section of an artery to be treated, a first, relatively small diameter balloon positioned distally in the treatment zone section, a second, relatively large diameter inflatable balloon positioned proximally within the treatment zone section of the catheter body in longitudinal alignment with the first balloon and with the catheter body, a first dilation lumen extending in fluid communication along the catheter body between a first dilation port and the first balloon, and a second dilation lumen extending in fluid communication along the catheter body between a second dilation port and the second balloon.
(b) introducing the treatment zone section of the elongate catheter body into a section of an artery to be treated,
(c) inflating the first balloon with a fluid to an extent sufficient to cause the first balloon dilate the artery in a manner defining a smooth, graduated transition between plaque on artery walls in the section of the artery being treated and plaque on an adjacent artery wall not being treated;
(d) inflating the second balloon with a fluid to an extent sufficient to cause the second balloon to dilate the artery in a manner defining a smooth, graduated transition between plaque on artery walls in the section of the artery being treated by the second balloon and plaque on artery walls in the section of the artery being treated by the first balloon;
(e) deflating the first and second balloons; and
(f) withdrawing the catheter body from the artery.

12. A method according to claim 11, and including the step of:
(a) providing a third, relatively small diameter balloon positioned most proximally in the treatment zone section in longitudinal alignment with the first balloon, the second balloon and the catheter body, the treatment zone section of the catheter body defining a fore and aft-tapered wall;
(b) inflating the third balloon with a fluid to an extent sufficient to cause the third balloon to dilate the artery in a manner defining a smooth, graduated transition between plaque on artery walls in the section of the artery being treated and plaque on an adjacent artery wall not being treated; and
(c) deflating the third balloon.

13. A method according to claim 11, and including the step of guiding the catheter body in the artery on a monorail guide wire.

14. A method according to claim 11, including the steps of inflating the small diameter balloon with a fluid at a predetermined relatively high pressure, and inflating the large diameter balloon with a fluid at a predetermined relatively low pressure.

15. A method according to claim 12, including the steps of inflating the first and third balloons with a liquid at a predetermined relatively high pressure, and inflating the second, large diameter balloon with a liquid at a predetermined relatively low pressure.

16. A method according to claim 11, and including the step of enclosing the multiple balloon expansion segment within a membrane, or covering adapted to expand as the balloons are inflated and contract as the balloons are deflated. This membrane further promotes the desired graduated contour.
